(19) United States
(12) Patent Application Publication
(10) Pub. No.: US 2018/0235292 A1
(43) Pub. Date: Aug. 23, 2018

(54) Aqua Exercise Glove

(71) Applicant: AyoDamola Akinyemi, Middletown, NY (US)

(72) Inventor: AyoDamola Akinyemi, Middletown, NY (US)

(21) Appl. No.: 15/900,173

(22) Filed: Feb. 20, 2018

Related U.S. Application Data
(60) Provisional application No. 62/461,148, filed on Feb. 20, 2017.

(51) Int. Cl. A41D 19/00; A63B 71/06; A63B 24/00; A63B 21/00; A63B 21/06; A63B 21/065; A63B 23/12; A41F 1/06; A61B 5/00; A61B 5/0205; A45F 3/20

(52) U.S. Cl. CPC A41D 19/0024; A61B 5/02438; A63B 24/0087; A63B 24/0062; A41D 19/002; A63B 21/0004; A63B 21/0602; A63B 21/065; A63B 21/4043; A63B 23/12; A41F 1/06; A41D 19/0037; A41D 19/0027; A61B 5/6806; A61B 5/0205; A45F 3/20; A63B 2225/09; A63B 2225/682; A63B 2230/06; A63B 2225/50; A63B 2230/30; A41D 2300/322; A63B 2220/803; A41D 2400/46; A41D 2600/10; A63B 2220/17; A63B 2209/00; A61B 2560/0214; A61B 2562/0219; A63B 71/0622

(57) ABSTRACT

An exercise glove which provides a use with portable water and a means of increasing resistance throughout a variety of exercise. The exercise glove includes a glove body and a liquid-storage container. The glove body includes a backhand portion, a palm portion, and an adjustable wrist band. The liquid-storage container includes a bladder body and a spout. The liquid-storage container stores liquid and is positioned adjacent to the backhand portion and the bladder body is mounted to the backhand portion. The spout provides a means of accessing the liquid within the bladder body and is externally and laterally connected to the bladder body. Additionally, the spout is in fluid communication with the bladder body. The exercise glove may also include a gyroscope, a microcontroller, a rechargeable battery, a wireless communication device, a heart-rate sensor, and a waterproof display screen to collect and convey various body characteristics to the user.

The current application claims a priority to the U.S. Provisional Patent application Ser. No. 62/461,148 filed on Feb. 20, 2017.

### FIELD OF THE INVENTION

The present invention relates generally to a fitness glove. More specifically, the present invention is a workout glove with an integrated liquid-storage container to weigh down the glove and to provide the wearer with water anytime during his or her exercise routine.

### BACKGROUND OF THE INVENTION

Exercise is essential to a healthy lifestyle. It is an object of the present invention to improve a user's workout experience, thereby increasing motivation to exercise. For effective and efficient workouts, it is important to stay hydrated throughout the workout. Staying hydrated promotes cardiovascular health, keeps the body cool, cleanses the body, and prevents dry mouth to name a few benefits. The present invention makes water and other similar liquids more accessible to the user even during his or her workout. More specifically, the present invention is an exercise glove with an integrated water pouch. Additionally, because the user wears the present invention, the weight of the liquid being stored by the present invention acts as a free weight to add additional resistance to the user during his or her workout. Thus, the present invention allows the user to perform full body movements and resultantly countless exercises. With the present invention, the user may perform a variety of exercises in order to build one's aerobic endurance, muscle strength, balance, and or flexibility.

The present invention incorporates a multitude of sensors in order to monitor the users' heart rate and blood pressure. Additionally, the present invention includes a pedometer feature. The aforementioned features provide the user with valuable information in order to ensure he or she is at in an optimal and healthy state during his or her workout routine.

### BRIEF DESCRIPTION OF THE DRAWINGS

FIG. 1 is a perspective view of the present invention.

FIG. 2 is a perspective view of the present invention in an exploded state.

FIG. 3 is a side perspective view of the present invention.

FIG. 4 is a detailed view of an alternative embodiment of the present invention, depicting a waterproof display screen.

FIG. 5 is an electronic schematic of the present invention.

FIG. 6 is an electric schematic of the present invention.

FIG. 7 is a schematic diagram of the present invention.

### DETAIL DESCRIPTIONS OF THE INVENTION

All illustrations of the drawings are for the purpose of describing selected versions of the present invention and are not intended to limit the scope of the present invention.

The present invention generally relates to exercise equipment. More specifically, the present invention is a multifunctioning fitness glove which simultaneously serves as an additional weight for the user and as a portable beverage container.

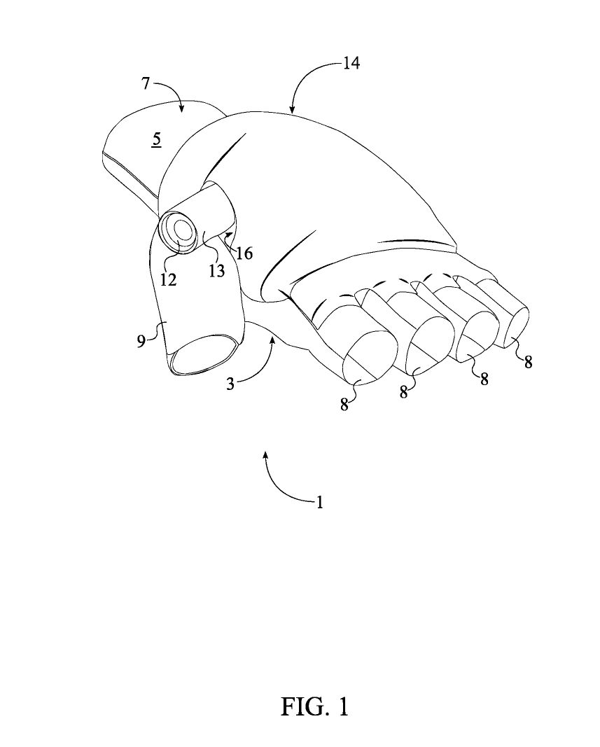

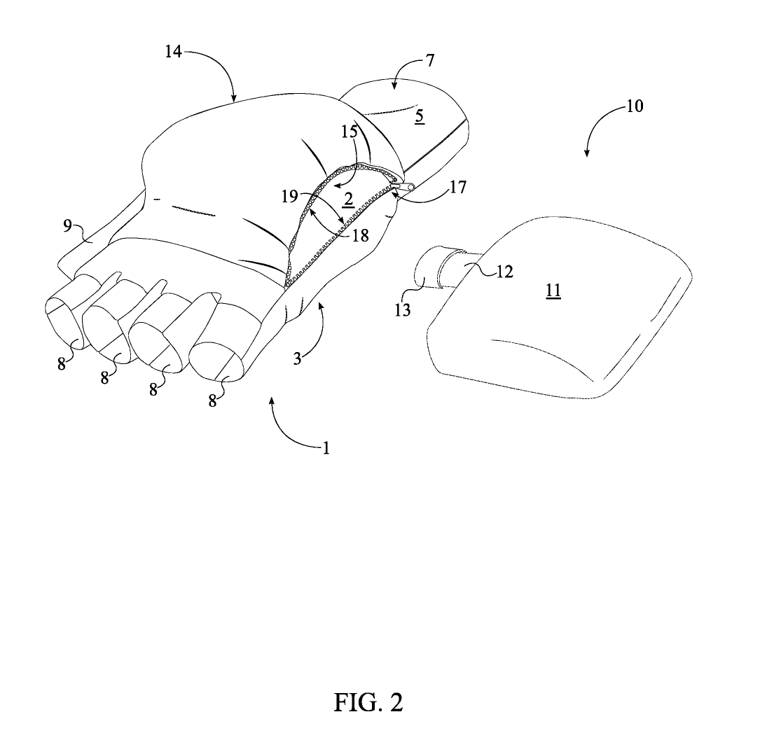

Referring to FIG. 1 and FIG. 2, in its simplest embodiment, the present invention comprises a glove body 1 and an at least one liquid-storage container 10. The glove body 1 is the structural body of a garment that is used to partially or fully cover the user's hand during various exercises and exercise routines. Similar to conventional designs, the glove body 1 comprise a backhand portion 2, a palm portion 3, a hand-receiving opening, and an adjustable wrist band 5. The backhand portion 2 is a piece of material designed to cover and protect the backhand of the user's hand. Complimentary, the palm portion 3 is a piece of material designed to cover and protect the palm of the user's hand. The backhand portion 2 and the palm portion 3 are positioned opposite to each other about the glove body 1 and, resultantly, outline the overall shape and profile of a hand. The hand-receiving opening is an oval or circular opening which allows the user to insert his or her hand into the glove body 1. The adjustable wrist band 5 wraps around the user's wrist and ensures that the present invention does not slip off of the user's hand. The adjustable wrist band 5 is an annular band that is preferably composed of an elastic material, although alternative compositions and designs may also be utilized. Additionally, the adjustable wrist band 5 may also include a means of controlling the diameter of the adjustable wrist band 5 such as a strap, hook-and-loop fastener, rope, or other similar mechanisms. The adjustable wrist band 5 is positioned about the hand-receiving opening and is perimetrically connected to the palm portion 3 and the backhand portion 2.

Referring to FIG. 1 and FIG. 2, the liquid-storage container 10 attaches to the glove body 1 and stores and disperses liquid, such as water. More specifically, the liquid-storage container 10 is positioned adjacent to the backhand portion 2, opposite the palm portion 3. The liquid-storage container 10 comprises a bladder body 11 and a spout 12. The bladder body 11 is an enclosed capsule/vessel designed to receive and contain a volume of liquid. The bladder body 11 is mounted to the backhand portion 2 in order to not interfere with the user's ability to grasp items and perform various exercises. The bladder body 11 may be mounted through a variety of means including, but not limited to, integration into the material of the backhand portion 2 and external attachment. The preferred bladder body 11 is composed of a flexible material such that the bladder body 11 may stretch to fit more or less liquid. Additionally, the bladder body 11 includes a refill hole with a removable cap for refilling purpose. The spout 12 is the element of the present invention through which the user consumes the liquid within the bladder body 11. More specifically, the spout 12 is a tubular extrusion through which liquid may be poured. The spout 12 is externally and laterally connected to the bladder body 11. Additionally, the spout 12 is in fluid communication with the bladder body 11 to allow for liquid to be dispersed from the bladder body 11. In one embodiment of the present invention, the spout 12 is either large enough for pouring water/liquid into the bladder body 11 or is removable for refill purposes.

Referring to FIG. 2, the bladder body 11 is mounted to the backhand portion 2 through a bladder-receiving pocket 14. Additional necessary components include a fastening mechanism 17 and a spout hole 16. The bladder-receiving pocket 14, the spout hole 16, and the fastening mechanism 17 allow for the removal of the bladder body 11 and the spout 12 for cleaning, replacement, and refill purposes. The bladder-receiving pocket 14 is a pouch-like compartment that is sized to receive and hold the bladder body 11. The bladder-receiving pocket 14 is adjacently integrated into the backhand portion 2 with a main opening 15 of the bladder-receiving pocket 14 being oriented away from the adjustable wrist band 5. This orientation of the main opening 15 may be implemented in a different orientation based on the needs and preferences of the user. The bladder-receiving pocket 14 may be stitched directly to the backhand portion 2, composed partly of the backhand portion 2, glued to the backhand portion 2, or any other similar fastening means. The spout hole 16 receives the spout 12, thus allowing a portion of the spout 12 to be positioned external to the bladder-receiving pocket 14. More specifically, the spout 12 laterally traverses into the bladder-receiving pocket 14. In order to mount the liquid-storage container 10 to the glove body 1, the bladder body 11 is positioned within the bladder-receiving pocket 14 with the spout 12 being positioned within the spout hole 16. The bladder-receiving pocket 14 is sized to fully enclose the bladder body 11, regardless of the amount of liquid within the bladder body 11.

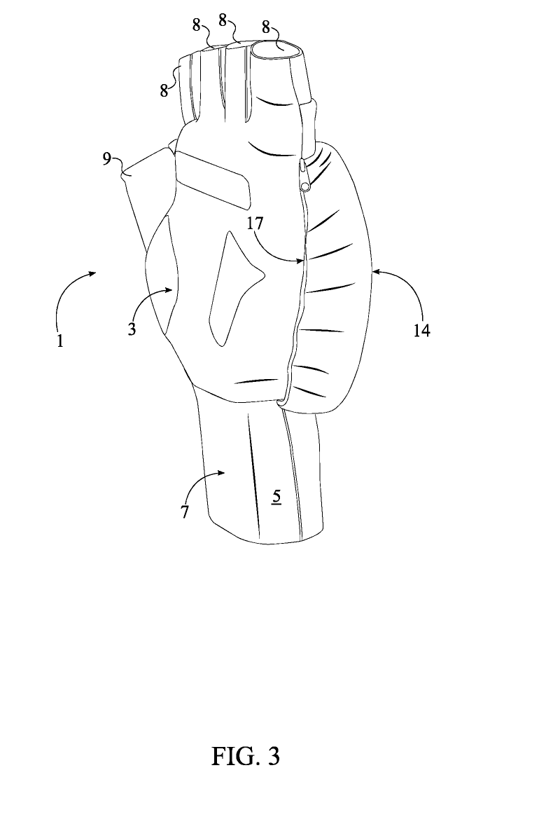

Referring to FIG. 2 and FIG. 3, the fastening mechanism 17 secures the bladder body 11 within the bladder-receiving pocket 14 by attaching the bladder-receiving pocket 14 to the backhand portion 2 at the main opening 15. The fastening mechanism 17 comprises a first interlocking element 18 and a second interlocking element 19. In relation to the glove body 1 and the bladder-receiving pocket 14, the fastening mechanism 17 is positioned adjacent to the main opening 15. The first interlocking element 18 is adjacently connected to the bladder-receiving pocket 14, about the main opening 15, while the second interlocking element 19 is adjacently connected to the backhand portion 2. In order to close off the main opening 15, the first interlocking element 18 is engaged to the second interlocking element 19. The fastening mechanism 17 can be, but is not limited to, hook-and-loop pads, magnets, buttons, and latches. In the preferred embodiment of the present invention, the fastening mechanism 17 is a zipper closing mechanism to ensure the liquid-storage container 10 remains mounted regardless of the activity or environmental factors such as sweat or water in or around the present invention.

In addition, the glove body 1 may further comprise an at least one finger sleeve 8 and a thumb sleeve 9. The at least one finger sleeve 8 is the portion of the glove body 1 that is used to cover the finger(s) of the user's hand. The finger sleeve 8 is a tubular piece of fabric sized and contoured to receive a specific finger of the user's hand. The finger sleeve 8 is positioned opposite to the adjustable wrist band 5, across the glove body 1. Additionally, the finger sleeve 8 is laterally integrated into the backhand portion 2 and the palm portion 3, similar to traditional glove designs. The thumb sleeve 9 is the portion of the glove body 1 which covers the thumb of the user's hand. The thumb sleeve 9 is a tubular piece of fabric sized and contoured to receive the thumb, and the thumb sleeve 9 is positioned in between the finger sleeve(s) 8 and the adjustable wrist band 5. Additionally, the thumb sleeve 9 is laterally integrated into the backhand portion 2 and the palm portion 3. In the preferred, embodiment, the present invention comprises four finger sleeves 8 and the thumb sleeve 9. The finger sleeve(s) 8 and or the thumb sleeve 9 may vary in length to meet the needs and preferences of the user.

Referring to FIG. 2, the liquid-storage container 10 further comprises a drinking nipple 13. The drinking nipple 13 allows water to flow out of the spout 12 based only when a suction is created around the drinking nipple 13 or a certain pressure is applied onto the drinking nipple 13. Essentially, no water or liquid is dispersed by the spout 12 unless the user applies a certain amount of pressure, usually with his or her mouth, onto the drinking nipple 13. The drinking nipple 13 is terminally connected to the spout 12, opposite to the bladder body 11. Resultantly, the drinking nipple 13 is in fluid communication with the bladder body 11 through the spout 12. A variety of designs and devices may be used as the drinking nipple 13.

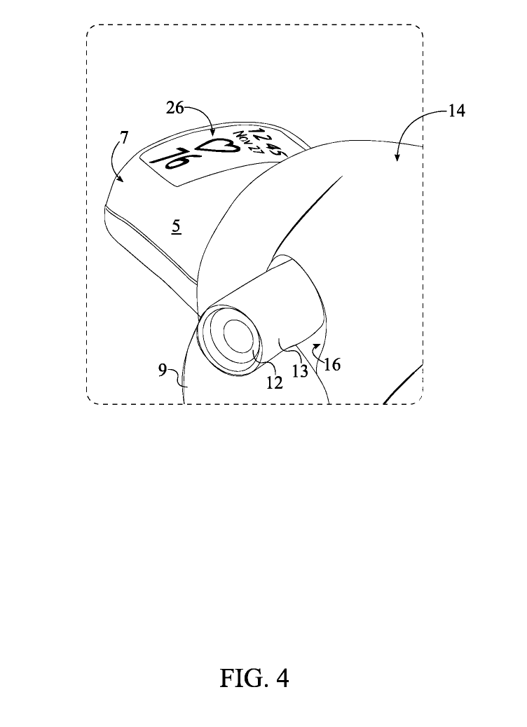

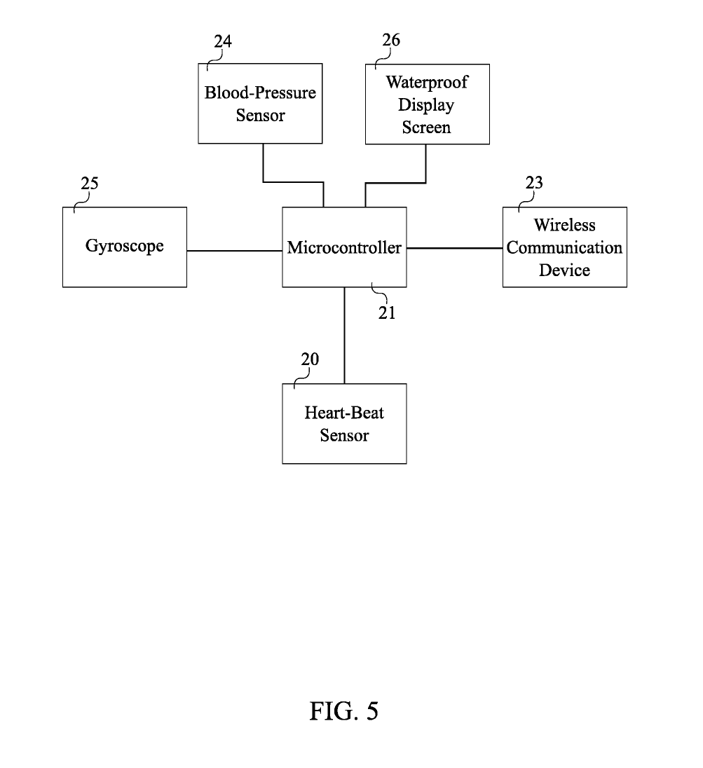

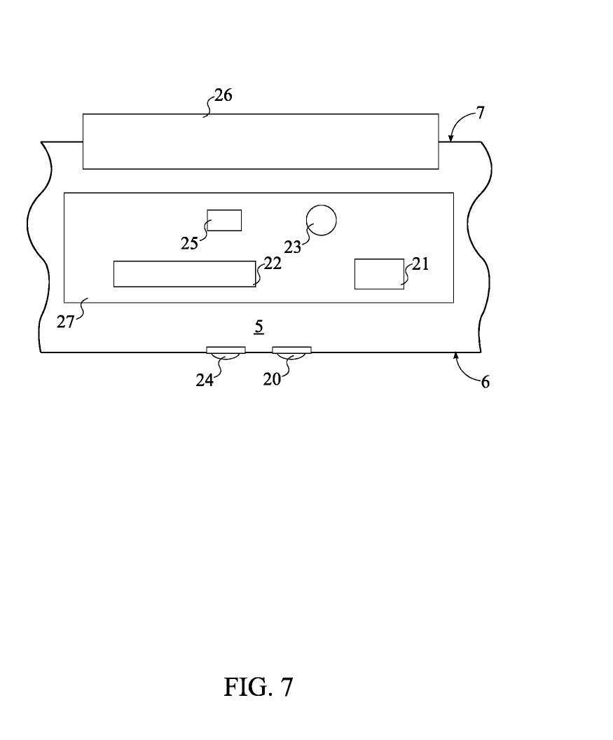

Referring to FIG. 4 and FIG. 7, the present invention further comprises a heart-rate sensor 20, a microcontroller 21, a rechargeable battery 22, a wireless communication device 23, a blood-pressure sensor 24, a gyroscope 25, and a waterproof display screen 26. The aforementioned components provide the user with valuable information about his or her physical health and fitness. Such information includes, but is not limited to, number of steps taken, heart rate, and blood pressure. Referring to FIG. 5, the microcontroller 21 is electronically connected to the heart-rate sensor 20, the wireless communication device 23, the blood-pressure sensor 24, the gyroscope 25, and the waterproof display screen 26. The microcontroller 21 manages and controls the data sent and received by the aforementioned components in order to provide the user with relevant information relating to his or her health/fitness. The microcontroller 21 is integrated into the adjustable wrist band 5, similar to traditional fitness bands.

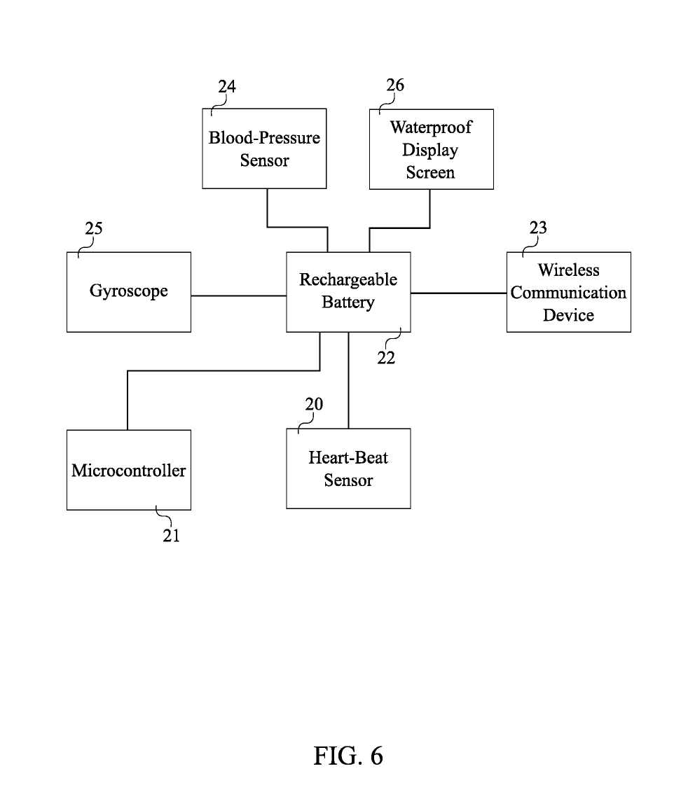

Referring to FIG. 6, the rechargeable battery 22 powers the aforementioned components. More specifically, the rechargeable battery 22 is electrically connected to the microcontroller 21, the wireless communication device 23, the heart-rate sensor 20, the blood-pressure sensor 24, the gyroscope 25, and the waterproof display screen 26. The rechargeable battery 22 is integrated into the adjustable wrist band 5, similar to traditional fitness bands. The rechargeable battery 22 may be removable or rechargeable through a port.

The wireless communication device 23 connects the present invention to an external computing device in order to send and receive information and commands for the present invention. Resultantly, the present invention may be paired and compatible with external health and fitness applications and programs to further track and monitor the user's health/fitness. The wireless communication device 23 is integrated into the adjustable wrist band 5, similar to traditional fitness bands.

The waterproof display screen 26 graphically displays information to the user directly while the present invention is worn. The waterproof display screen 26 is preferably integrated into an outer surface 7 of the adjustable wrist band 5, directly in the user's view. In one embodiment of the present invention, the waterproof display screen 26 is a touch screen, thus allowing the user to interact with the present invention through the waterproof display screen 26.

The heart-rate sensor 20 monitors and records the user's hear rate. The heart rate is one of the metrics that directly indicates cardiovascular activity and is important to know during a physical activity. The heart-rate sensor 20 is integrated into an inner surface 6 of the adjustable wrist band 5 such that the heart-rate sensor 20 is directly pressed against the skin of the user, this is necessary for measuring and recording the heart-beat. The blood-pressure sensor 24 monitors and records the user's blood pressure. Similar to the heart-rate sensor 20, the blood-pressure sensor 24 is integrated into the inner surface 6 such that the sensor is directly next to the user's skin. It is preferred that the blood-pressure sensor 24 and the heart-rate sensor 20 are both waterproof to ensure functionality in any environment. The gyroscope 25 measures orientation and angular velocity. In the present invention, the gyroscope 25 is used to count the number of steps taken by the user. The gyroscope 25 is integrated into the adjustable wrist band 5, similar to traditional fitness bands.

In one embodiment, the present invention further comprises a waterproof housing 27. The waterproof housing encloses and protects the electronic components of the present invention and prevents water damage. More specifically, the wireless communication device 23, the gyroscope 25, the microcontroller 21, and the rechargeable battery 22 are mounted within the waterproof housing 27. The waterproof housing 27 is integrated into the adjustable wrist band 5.

In one embodiment, the present invention further comprises a plurality of light emitting diodes (LEDs). The plurality of LEDs is integrated into the glove body 1, preferably into the adjustable wrist band 5, in order to indicate a variety of information including, but not limited to, an alarm time, a water break, high blood pressure, high and a preset hear rate, specific number of steps reached, and other similar information.

The present invention provides the user with a weighted glove(s), a water source, and acts as a fitness band altogether. Resultantly, the user may perform a variety of exercises with an additional weight stress for increased physical strain while simultaneously keeping his or her hands free to grasp, punch, and generally move freely, contrary to traditional weights. When the user is thirsty, the user may simply drink from the drinking nipple 13. In order to replenish/refill the water, or other similar liquids, the user unzips or unlocks the fastening mechanism 17 and removes the liquid-storage container 10 for refill or replace purposes. Additionally, the user is provided with a plethora of information during and or after the exercise routine through the waterproof display screen 26 or through an external computing device that is wirelessly connected to the present invention through the wireless communication device 23.

Although the invention has been explained in relation to its preferred embodiment, it is to be understood that many other possible modifications and variations can be made without departing from the spirit and scope of the invention as hereinafter claimed.

What is claimed is:

1. An aqua exercise glove comprises:
a glove body;
an at least one liquid-storage container;
the glove body comprises a backhand portion, a palm portion, and an adjustable wrist band;
the liquid-storage container comprises a bladder body and a spout;
the backhand portion the palm portion being positioned opposite to each other about the glove body;
the adjustable wrist band being perimetrically connected to the palm portion and the backhand portion;
the liquid-storage container being positioned adjacent to the backhand portion, opposite to the palm portion;
the bladder body being mounted to the backhand portion;
the spout being externally and laterally connected to the bladder body; and
the spout being in fluid communication with the bladder body.

2. The aqua exercise glove as claimed in claim 1 comprises:
a bladder-receiving pocket;
a spout hole;
a fastening mechanism;
the fastening mechanism comprises a first interlocking element and a second interlocking element;
the liquid-storage container being mounted to the backhand portion through the bladder-receiving pocket;
the bladder-receiving pocket being adjacently integrated into the backhand portion;
a main opening of the bladder-receiving pocket being oriented away from the adjustable wrist band;
the first interlocking element being adjacently connected to the bladder-receiving pocket, about the main opening;
the second interlocking element being adjacently connected to the backhand portion;
the bladder body being positioned within the bladder-receiving pouch;
the spout hole laterally traversing into the bladder-receiving pouch; and
the spout being positioned within the spout hole.

3. The aqua exercise glove as claimed in claim 2, wherein the first interlocking element and the second interlocking element are engaged with each other.

4. The aqua exercise glove as claimed in claim 2, wherein the fastening mechanism is a zipper closing mechanism.

5. The aqua exercise glove as claimed in claim 1 comprises:
the glove body further comprises an at least one finger sleeve and a thumb sleeve;
the finger sleeve being positioned opposite to the adjustable wrist band, across the glove body;
the finger sleeve being laterally integrated into the backhand portion and the palm portion;
the thumb sleeve being positioned in between the finger sleeve and the adjustable wrist band; and
the thumb sleeve being laterally integrated into the backhand portion and the palm portion.

6. The aqua exercise glove as claimed in claim 1 comprises:
the liquid-storage container further comprises a drinking nipple;
the drinking nipple being terminally connected to the spout, opposite to the bladder body; and
the drinking nipple being in fluid communication with the bladder body through the spout.

7. The aqua exercise glove as claimed in claim 1 comprises:
a heart-rate sensor;
a microcontroller;
a rechargeable battery;
a wireless communication device;
the microcontroller, the rechargeable battery, and the wireless communication device being integrated into the adjustable wrist band;
the rechargeable battery being electrically connected to the microcontroller, the wireless communication device, and the heart-rate sensor;
the microcontroller being electronically connected to the heart-rate sensor and the wireless communication device; and
the heart-rate sensor being integrated to an inner surface of the adjustable wrist band.

8. The aqua exercise glove as claimed in claim 6, wherein the hear-rate sensor is waterproof.

9. The aqua exercise glove as claimed in claim 1 comprises:
a blood-pressure sensor;
a microcontroller;
a rechargeable battery;
a wireless communication device;
the microcontroller, the rechargeable battery, and the wireless communication device being integrated into the adjustable wrist band;
the rechargeable battery being electrically connected to the microcontroller, the wireless communication device, and the blood-pressure sensor;
the microcontroller being electronically connected to the blood-pressure sensor and the wireless communication device; and
the blood-pressure sensor being integrated to an inner surface of the adjustable wrist band.

10. The aqua exercise glove as claimed in claim 8, wherein the blood-pressure sensor is waterproof.

11. The aqua exercise glove as claimed in claim 1 comprises:
a gyroscope;
a microcontroller;
a rechargeable battery;
a wireless communication device;
the microcontroller, the rechargeable battery, the wireless communication device, and the gyroscope being integrated into the adjustable wrist band;
the rechargeable battery being electrically connected to the microcontroller, the wireless communication device, the gyroscope, and the the heart-rate sensor;
the microcontroller being electronically connected to the heart-rate sensor, the wireless communication device, and the gyroscope; and
the heart-rate sensor being integrated to an inner surface of the adjustable wrist band.

12. The aqua exercise glove as claimed in claim 1 comprises:
a heart-rate sensor;
a microcontroller;
a rechargeable battery;
a wireless communication device;
a waterproof display screen;
the microcontroller, the rechargeable battery, and the wireless communication device being integrated into the adjustable wrist band;
the rechargeable battery being electrically connected to the microcontroller, the wireless communication device, the heart-rate sensor, and the waterproof display screen;
the microcontroller being electronically connected to the heart-rate sensor, the wireless communication device, and the waterproof display screen;
the heart-rate sensor being integrated into an inner surface of the adjustable wrist band; and
the waterproof display screen being integrated into an outer surface of the adjustable wrist band.

13. The aqua exercise glove as claimed in claim 1 comprises:
a blood-pressure sensor;
a microcontroller;
a rechargeable battery;
a wireless communication device;
a waterproof display screen;
the microcontroller, the rechargeable battery, and the wireless communication device being integrated into the adjustable wrist band;
the rechargeable battery being electrically connected to the microcontroller, the wireless communication device, the waterproof display screen, and the blood-pressure sensor;
the microcontroller being electronically connected to the blood-pressure sensor, the waterproof display screen, and the wireless communication device;
the blood-pressure sensor being integrated to an inner surface of the adjustable wrist band; and
the waterproof display screen being integrated into an outer surface of the adjustable wrist band.

14. The aqua exercise glove as claimed in claim 1 comprises:
a gyroscope;
a microcontroller;
a rechargeable battery;
a wireless communication device;
a waterproof display screen;
the microcontroller, the rechargeable battery, the wireless communication device, and the gyroscope being integrated into the adjustable wrist band;
the rechargeable battery being electrically connected to the microcontroller, the wireless communication device, the gyroscope, the waterproof display screen, and the heart-rate sensor;
the microcontroller being electronically connected to the waterproof display screen, the wireless communication device, and the gyroscope;
the heart-rate sensor being integrated to an inner surface of the adjustable wrist band; and
the waterproof display screen being integrated into an outer surface of the adjustable wrist band.

15. The aqua exercise glove as claimed in claim 1 comprises:
a waterproof housing;
a heart-rate sensor;
a gyroscope;
a microcontroller;
a rechargeable battery;
a wireless communication device;
a waterproof display screen;
a blood-pressure sensor;
the rechargeable battery being electrically connected to the microcontroller, the wireless communication device, the gyroscope, the waterproof display screen, the heart-rate sensor, the hear-rate sensor, and the blood-pressure sensor;
the microcontroller being electronically connected to the waterproof display screen, the wireless communication device, the gyroscope, the heart-rate sensor, and the blood-pressure sensor;
the waterproof display screen being integrated into an outer surface of the adjustable wrist band;
the heart-rate sensor being integrated to an inner surface of the adjustable wrist band;
the blood-pressure sensor being integrated to the inner surface;
the gyroscope, the microcontroller, the wireless communication device, and the rechargeable battery being mounted within the waterproof housing; and
the waterproof housing being integrated into the adjustable wrist band.
